(19) United States
(12) Patent Application Publication
(10) Pub. No.: US 2020/0061578 A1
(43) Pub. Date: Feb. 27, 2020

(54) ABSORBENT POLYMERS, AND METHODS OF PRODUCING THEREOF AND USES THEREOF

(71) Applicant: Novomer, Inc., Boston, MA (US)

(72) Inventors: Sadesh H. SOOKRAJ, Cambridge, MA (US); Alexander TSEITLIN, Acton (CA); Han LEE, Chicago, IL (US); Konstantin A. POKROVSKI, Boston, MA (US)

(73) Assignee: Novomer, Inc., Boston, MA (US)

(21) Appl. No.: 16/346,856

(22) PCT Filed: Oct. 31, 2017
(86) PCT No.: PCT/US17/59243

§ 371 Date: May 1, 2019

Related U.S. Application Data
(60) Provisional application No. 62/416,623, filed on Nov. 2, 2016.

(51) Int. Cl. B01J 20/26; C08G 81/02; C08J 3/24; B01J 20/28; B01J 20/30; A61L 15/26; A01C 1/06

(52) U.S. Cl. CPC B01J 20/267; C08G 81/027; C08J 3/246; C08G 2230/00; B01J 20/3085; A61L 15/26; A01C 1/06; B01J 20/28033

(57) ABSTRACT

Provided herein are absorbent polymers produced from beta-propiolactone, and methods of producing such polymers. These absorbent polymer may be cross-linked. The beta-propiolactone may be derived from ethylene oxide and carbon monoxide. The absorbent polymer may be bio-based and/or bio-degradable. The absorbent polymers may be used for diapers, adult incontinence products, and feminine hygiene products, as well as for agricultural applications.

### CROSS-REFERENCE TO RELATED APPLICATION

This application claims priority to U.S. Provisional Application No. 62/416,623, filed on Nov. 2, 2016, which is hereby incorporated by reference in its entirety.

### FIELD

The present disclosure relates generally to polymeric materials, and more specifically to polymeric materials suitable for use as adsorbent materials, and methods of producing thereof.

### BACKGROUND

Superabsorbent polymers are polymeric materials that can absorb and retain huge amounts of water or aqueous solutions. Such polymeric materials are used extensively for the manufacture of diapers, adult incontinence products, and feminine hygiene products, as well as well as in agricultural applications.

Superabsorbent polymers are commonly produced from polymerization of acrylic acid. However, due to volatile acrylic acid price and supply deficit, there is a desire in the art to produce polymeric materials with adsorbent properties from alternative sources. In particular, there is a need in the art to produce bio-based, bio-degradable polymeric materials with adsorbent properties, obtained from renewable sources.

### BRIEF SUMMARY

Provided herein are polymeric materials with adsorbent properties, and methods of producing thereof, that addresses the need in the art. Such polymeric materials may be obtained from beta-propiolactone, which may be derived from renewable sources, such as bio-based ethylene oxide and carbon monoxide.

In some aspects, provided is a method of producing a cross-linked polymer, comprising combining beta-propiolactone and a cross-linker to produce the cross-linked polymer, wherein the cross-linked polymer comprises a partially neutralized polyacrylic acid backbone and a plurality of polypropiolactone side chains, and cross-linking moieties. In some variations of the foregoing, the polypropiolactone side chains independently have a structure of formula —(CH₂CH₂(C═O)—O)ₙ⁻M⁺, wherein: n is an integer from 1 to 10 inclusive; and M⁺ is an alkali metal, a cross-linking moiety, or H⁺.

In certain aspects, provided is a method of producing a cross-linked polymer, comprising combining beta-propiolactone and a cross-linker in the presence of a metal cation to produce the cross-linked polymer, wherein the cross-linked polymer comprises a partially neutralized polyacrylic acid backbone and a plurality of polypropiolactone side chains, and cross-linking moieties. In certain variations, the source of the metal cation is a metal salt. For example, in one variation, the metal salt may be a metal acrylate.

In certain aspects, provided is a method of producing a cross-linked polymer, comprising reacting a low molecular weight polypropiolactone with a radical polymerization initiator and a cross-linker, wherein the low molecular weight polypropiolactone has a formula CH₂═CH₂—(C═O)—O—(CH₂CH₂(C═O)—O)ₙ⁻M⁺, wherein n is an integer from 1 to 10 inclusive; and M⁺ is an alkali metal, a cross-linking moiety, or H⁺.

In other aspects, provided is a polymer produced according to any of the methods described herein.

In some aspects, provided is a polymer comprising a poly(sodium acrylate/acrylic acid) backbone and a plurality of polypropiolactone side chains connected to the backbone. In some embodiments, the polymer is cross-linked. In some variations of the foregoing, the polymer is bio-based and/or bio-degradable.

The polymers described herein, or produced according to the methods described herein, may be suitable for use as an absorbent article (e.g., for diapers, adult incontinence products, or feminine hygiene products) or as agricultural products (e.g., for agricultural materials, and seed coatings).

### DESCRIPTION OF THE FIGURES

The present application can be best understood by reference to the following description taken in conjunction with the accompanying figures, in which like parts may be referred to by like numerals.

FIGS. 1-3 depict exemplary processes to produce the polymer described herein from beta-propiolactone.

FIG. 4 depicts an exemplary process to produce beta-propiolactone from ethylene oxide and carbon monoxide.

FIG. 5 depicts an exemplary polymer comprising a poly(sodium acrylate/acrylic acid) backbone and a plurality of polypropiolactone side chains connected to the backbone.

FIG. 6 depicts an exemplary polymer comprising a poly(sodium acrylate/acrylic acid) backbone and a plurality of cross-linked polypropiolactone side chains connected to the backbone. The type cross-linking in such polymer will depend on the cross-linker used.

FIG. 7A depicts an exemplary cross-linked polymer in which N,N′-methylenebis(acrylamide) is the cross-linker.

FIG. 7B depicts an exemplary cross-linked polymer in which ethylene carbonate is the cross-linker.

FIG. 7C depicts an exemplary cross-linked polymer in which aluminum acrylate is the cross-linker.

FIG. 7D depicts an exemplary cross-linked polymer in which ethylene glycol diglycidyl ether is the cross-linker.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein are polymers that have absorbent properties. In some aspects, such polymers are produced from beta-propiolactone. The beta-propiolactone may be produced from carbonylation of ethylene oxide. When the ethylene oxide and carbon monoxide are obtained from renewable sources, the polymers described herein may be bio-based polymers. Moreover, the polymers described herein may be biodegradable. Such superabsorbent polymers may be used for diapers, adult incontinence products, and feminine hygiene products, maintaining or improving the performance of such products.

The methods of producing such absorbent polymers, and the structure and properties of such absorbent polymers are described in further detail below.

### Methods of Producing Absorbent Polymers

In some aspects, provided herein are polymers or polymer compositions produced from beta-propiolactone. Such polymers comprise a poly(sodium acrylate/acrylic acid) backbone and a plurality of polypropiolactone side chains connected to the backbone.

In some embodiments, provided is a method of producing a polymer composition, comprising combining beta-propiolactone and a cross-linker. The polymer composition comprises a cross-linked polymer.

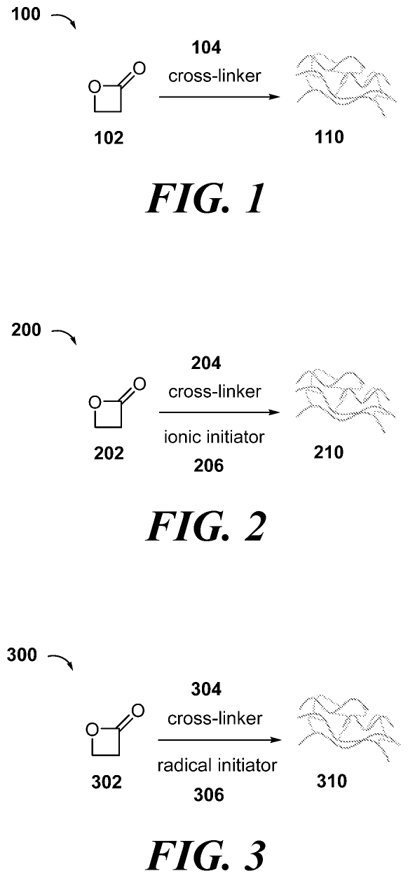

With reference to FIG. 1, process 100 is an exemplary process to produce cross-linked polymer 110 from beta-propiolactone 102 and cross-linker 104. The resulting cross-linked polymer 110 may comprise a partially neutralized polyacrylic acid backbone and a plurality of polypropiolactone side chains, and cross-linking moieties.

In some variations, the polypropiolactone side chains independently have a structure of formula —(CH₂CH₂(C═O)—O)ₙ⁻M⁺, wherein:

n is an integer from 1 to 10 inclusive; and

M⁺ is an alkali metal, a cross-linking moiety, or H⁺.

The length of the polypropiolactone side chains may vary and affect the absorbency of the polymer.

In some variations, the cross-linking moieties connect carboxylic end groups of at least a portion of the polypropiolactone side chains. In other variations, the cross-linking moieties connect neutralized carboxylate groups of at least a portion of the polypropiolactone side chains. In yet other variations, the cross-linking moieties connect at least a portion of the partially neutralized polyacrylic acid backbone.

In other embodiments, provided is a method of producing a cross-linked polymer, comprising combining beta-propiolactone, a cross-linker and an initiator. In some variations, the initiator is an ionic initiator. Thus, in some variations, with reference to FIG. 2, process 200 is an exemplary process to produce cross-linked polymer 210 from beta-propiolactone 202, cross-linker 204, and ionic initiator 206.

In other variations, the initiator is a radical initiator. Thus, in some variations, with reference to FIG. 3, process 300 is an exemplary process to produce cross-linked polymer 310 from beta-propiolactone 302, cross-linker 304, and radical initiator 306.

It should be generally understood that, in other exemplary variations, processes 100, 200, or 300 may include one or more additional reagents and/or one or more additional steps. For example, in some variations, a solvent may be used for the polymerization reaction. In other variations, the polymerization reaction is performed neat. In yet other variations, processes 100, 200, or 300 may further include increasing the cross-linking of the polymer. For example, in one variation, cross-linked polymer 110, 210 or 310 is combined with additional cross-linker(s) to increase surface cross-linking of the polymer.

In other embodiments, provided is a method of producing a cross-linked polymer, comprising reacting a low molecular weight polypropiolactone with a radical polymerization initiator and a cross-linker,

wherein the low molecular weight polypropiolactone has a formula CH₂═CH₂—(C═O)—O—(CH₂CH₂(C═O)—O)ₙ⁻M⁺,
wherein n is an integer from 1 to 10 inclusive; and
M⁺ is an alkali metal, a cross-linking moiety, or H.

In some variations of the foregoing embodiment, the low molecular weight polypropiolactone may be obtained from polymerizing beta-propiolactone.

The beta-propiolactone, cross-linker, and initiators are described in further detail below.

Beta-Propiolactone

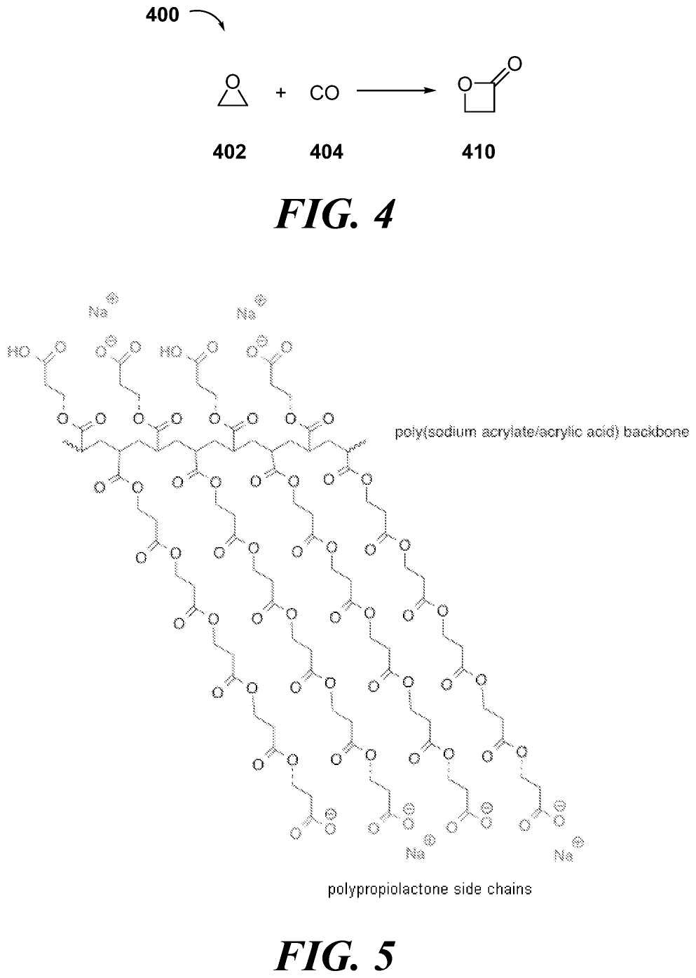

Beta-propiolactone may be produced by any suitable methods or techniques known in the art. For example, in some variations, with reference to FIG. 4, beta-propiolactone 410 is produced from ethylene oxide 402 and carbon monoxide 404. The ethylene oxide undergoes carbonylation in the presence of a carbonylation catalyst and optionally a solvent.

Thus, in some aspects, provided is a method of producing a cross-linked polymer, comprising: carbonylating ethylene oxide to produce beta-propiolactone; and combining the beta-propiolactone and a cross-linker to produce the cross-linked polymer. In some variations, the method comprises: combining ethylene oxide, carbon monoxide, a carbonylation catalyst and optionally a solvent to produce beta-propiolactone; and combining the beta-propiolactone and a cross-linker to produce the cross-linked polymer. In one variation, the method comprises: combining ethylene oxide, carbon monoxide, a carbonylation catalyst and a solvent to produce beta-propiolactone; and combining the beta-propiolactone and a cross-linker to produce the cross-linked polymer.

The beta-propiolactone may be isolated prior to polymerization to produce the polymers described herein. Thus, in some variations, provided is a method of producing a cross-linked polymer, comprising: carbonylating ethylene oxide to produce beta-propiolactone; isolating at least a portion of the beta-propiolactone produced, and combining the isolated beta-propiolactone and a cross-linker to produce the cross-linked polymer. In some variations, the method comprises: combining ethylene oxide, carbon monoxide, a carbonylation catalyst and optionally a solvent to produce beta-propiolactone; isolating at least a portion of the beta-propiolactone produced, and combining the isolated beta-propiolactone and a cross-linker to produce the cross-linked polymer. In one variation, the method comprises: combining ethylene oxide, carbon monoxide, a carbonylation catalyst and a solvent to produce beta-propiolactone; isolating at least a portion of the beta-propiolactone produced, and combining the isolated beta-propiolactone and a cross-linker to produce the cross-linked polymer.

In some variations of the foregoing, the carbon monoxide is provided in gaseous form. In other variations of the foregoing, the ethylene oxide is provided in gaseous form. In certain variations, gaseous ethylene oxide is converted to liquid form and combined with a solvent, a carbonylation catalyst and gaseous carbon monoxide in the reactor.

Any suitable carbonylation catalysts may be used to produce the beta-propiolactone. For example, in some variations, the carbonylation catalyst comprises a metal carbonyl compound. In certain variations, the carbonylation catalyst is a solid-supported metal carbonyl compound. Suitable carbonylation catalysts are described in, for example, WO 2010/118128. In some variations, the carbonylation catalyst comprises [(TPP)Al][Co(CO)₄], [(ClTPP)Al][Co(CO)₄], [(TPP)Cr][Co(CO)₄], [(ClTPP)Cr][Co(CO)₄], [(salcy)Cr][Co(CO)₄], [(salph)Cr][Co(CO)₄], or [(salph)Al][Co(CO)₄]. It should generally be understood that “TPP” refers to tetraphenylporphyrin; “ClTPP” refers to meso-tetra(4-chlorophenyl)porphyrin); “salcy” refers to (N, N′-bis(3,5-di-tert-butylsalicylidene)-1,2-diaminocyclohexane); and “salph” refers to (N, N′-bis(salicylidene)-o-phenylenediamine).

Any suitable solvents may be used to produce the beta-propiolactone. In some variations, the solvent comprises an ether solvent. In one variation, the solvent comprises tetrahydrofuran.

In one variation, the method comprises:

providing gaseous ethylene oxide;

converting gaseous ethylene oxide under suitable pressure conditions to produce liquid ethylene oxide;

combining liquid ethylene oxide with a solvent, a carbonylation catalyst and gaseous carbon monoxide to produce beta-propiolactone;

isolating at least a portion of the beta-propiolactone produced;

combining the isolated beta-propiolactone and a cross-linker to produce the cross-linked polymer.

Cross-Linkers

Various cross-linkers may be used in the methods described herein. Any combinations of the cross-linkers described herein may also be used.

In some embodiments, the cross-linker comprises an acrylamide compound, a metal acrylate compound, an organic carbonate compound, a diglycidyl compound, or a vinyl-organic compound comprising two or more vinyl groups.

In other embodiments, the cross-linker comprises a silane compound. In one embodiment, the silane compound has a structure of formula Y₃SiR^{a}N⁺R¹R²R³X⁻, wherein:

Y is a hydrolyzable radical;

R^{a} is a divalent hydrocarbon radical;

each of R¹, R² and R³ is independently:
a saturated or unsaturated hydrocarbon radical, or
a saturated or unsaturated organic radical comprising carbon, hydrogen, and at least one heteroatom selected from the group consisting of oxygen, sulfur and nitrogen; and

X⁻ is an anion.

In some variations of the silane compound, R^{a} is a divalent hydrocarbon radical with 1 to 6 carbon atoms. In certain variations of the silane compound, each of R¹, R² and R³ is independently a saturated or unsaturated organic radical comprising (i) carbon, hydrogen and oxygen, (ii) carbon, hydrogen, and sulfur, or (iii) or carbon, hydrogen and nitrogen. In one variation, each of R¹, R² and R³ is independently a saturated or unsaturated organic radical consisting of (i) carbon, hydrogen and oxygen, (ii) carbon, hydrogen, and sulfur, or (iii) or carbon, hydrogen and nitrogen.

In other variations of the silane compound, X⁻ is a halide, acetate or tosylate. In some variations, X⁻ is chloride, bromide, fluoride or iodide. In another variation, X⁻ is acetate. In yet another variation, X⁻ is tosylate.

In other embodiments, the cross-linker has at least two functional groups that can react with the carboxyl, carboxylate, vinyl or other reactive groups in the polymer chain to cross-link polymer chains on or in the vicinity of the surface of the polymer particles.

In some variations, the cross-linker is an organic compound comprising two or more vinyl groups. In other variations, the cross-linker is an organic compound comprises a Group 2, 3, or 4 metal cation. In yet other variations, the cross-linker is. an organic carbonate. In yet other variations, the cross-linker is an organic compound comprising two or more glycidyl groups.

In other embodiments, the cross-linker comprises a polyol or a polyglycidyl ether.

In yet other embodiments, the cross-linker comprises a polysaccharide.

In some variations, the cross-linker is ethyleneglycol dimethacrylate, diethyleneglycol diacrylate, allylmethacrylate, 1,1,1-trtimethylpropane triacrylate, triallylamine, tetraallyoxyethane, N,N′-methylenebis(acrylamide), aluminum acrylate, ethylene carbonate, or ethylene glycol diglycidyl ether. In one variation, the cross-linker is N,N′-methylenebis(acrylamide). In another variations, the cross-linker is ethylene carbonate. In yet another variations, the cross-linker is aluminum acrylate. In yet another variations, the cross-linker is ethylene glycol diglycidyl ether.

Initiators

In one variation, the initiator is an ionic initiator and/or a radical initiator. Any combinations of the initiators described herein may also be used.

For example, with reference to FIG. 2, process 200 is an exemplary process to produce cross-linked polymer 210 from beta-propiolactone 202, cross-linker 204, and ionic initiator 206.

In some variations, the ionic initiator comprises a salt of an alkali metal or a salt of an alkali-earth metal. In certain variations, the ionic initiator comprises a carboxylate salt of an alkali metal, or a salt of an alkali-earth metal. In one variations, wherein the ionic initiator is a salt of an alkali metal.

In other variations, the ionic initiator has a structure of formula CH₂═CH₂CO₂⁻Z⁺, wherein Z⁺ is an alkali metal, an alkali earth metal, ammonium, a quaternary ammonium cation, or phosphonium. In certain variations, the ionic initiator has a structure of formula CH₂═CH₂CO₂⁻Z⁺, wherein Z⁺ is a quaternary ammonium cation. In one variation, the quaternary ammonium cation is a lower alkyl quaternary ammonium cation.

In other variations, the ionic initiator is sodium acrylate, or potassium acrylate. In certain variations, the ionic initiator is a methacrylate. In one variation, the ionic initiator is sodium methacrylate, or potassium methacrylate.

In other example, with reference to FIG. 3, process 300 is an exemplary process to produce cross-linked polymer 310 from beta-propiolactone 302, cross-linker 304, and radical initiator 306.

In some variations, the radical initiator comprises a peroxide, a persulfate, or an azo compound. In other variations, the radical initiator is a redox initiator. In certain variations, the radical initiator comprises a hydroperoxide. In one variation, the radical initiator comprises hydrogen peroxide.

Additional Monomeric Compounds

The beta-propiolactone and the cross-linker, and optionally the initiators, may be further combined with an additional monomeric compound. Thus, in some embodiments, provided is a method of producing a cross-linked polymer, comprising combining beta-propiolactone, a cross-linker, optionally an initiator, and an additional monomeric compound to produce the cross-linked polymer.

In other embodiments, provided is a method of producing a cross-linked polymer, comprising reacting a low molecular weight polypropiolactone with a radical polymerization initiator, a cross-linker, and an additional monomeric compound,

wherein the low molecular weight polypropiolactone has a formula CH₂═CH₂—(C═O)—O—(CH₂CH₂(C═O)—O)ₙ⁻M⁺,
wherein n is an integer from 1 to 10 inclusive; and
M⁺ is an alkali metal, a cross-linking moiety, or H.

In some variations, the additional monomeric compound is an organic compound comprising at least one vinyl group. In other variations, the additional monomeric compound is an optionally substituted acrylic acid, or a carbohydrate, or any combination thereof. In one variation, the additional monomeric compound is methacrylic acid.

### Absorbent Polymers

In some aspects, provided are polymers produced according to any of the methods described herein. In other aspects, provided is a polymer comprising a poly(sodium acrylate/acrylic acid) backbone and a plurality of polypropiolactone side chains connected to the backbone. An example of such polymer is depicted in FIG. 5.

In some variations, the polypropiolactone side chains independently have a structure of formula —(CH₂CH₂(C═O)—O)ₙ⁻M⁺, wherein:

n is an integer from 1 to 100 inclusive; and

M⁺ is an alkali metal, a cross-linking moiety, or H.

In certain variations of the foregoing, n is an integer from 1 to 50, 1 to 40, 1 to 30, 1 to 20, or 1 to 10 inclusive.

In certain variations of the foregoing, M⁺ is an alkali metal. In one variation, M⁺ is Na⁺ or K⁺, or a combination thereof. In other variations, M⁺ is H. In yet other variations, M⁺ is an alkali metal, a cross-linking moiety. For example, M⁺ may be any of the cross-linking moieties described herein in cationic form.

In some variations, the polymers described herein are cross-linked. In other aspects, provided is a polymer comprising a partially neutralized polyacrylic acid backbone and a plurality of polypropiolactone side chains, and cross-linking moieties.

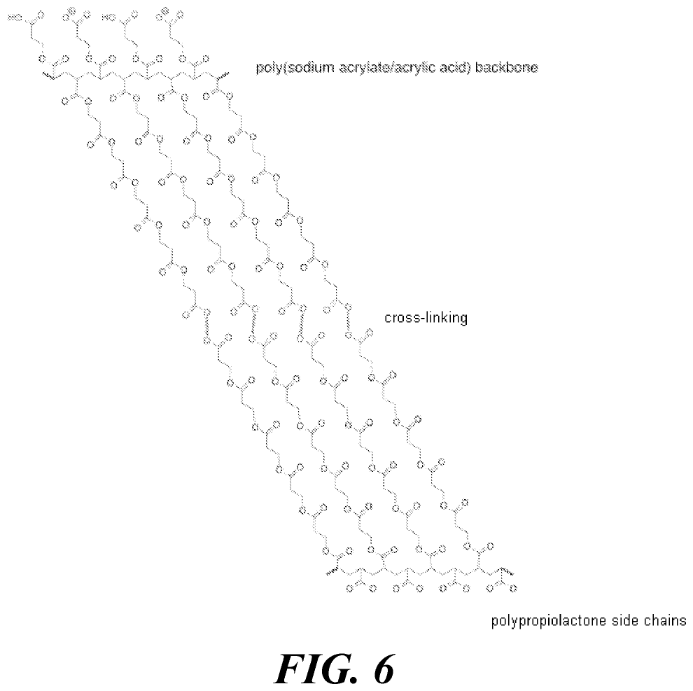

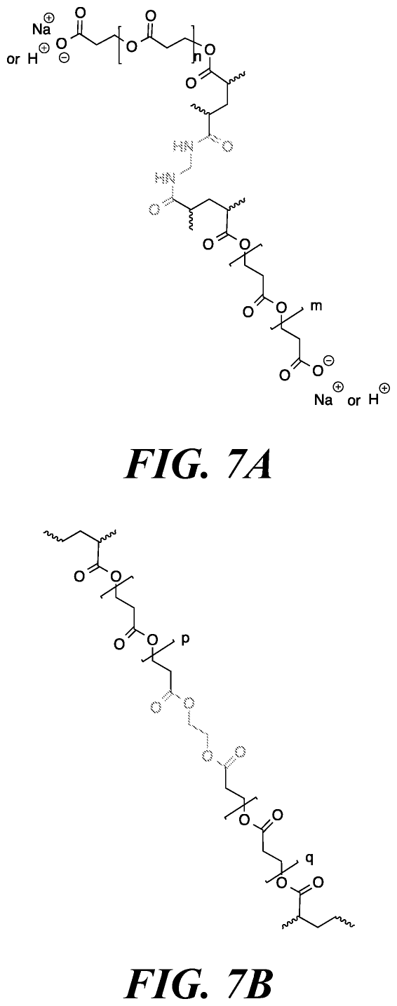

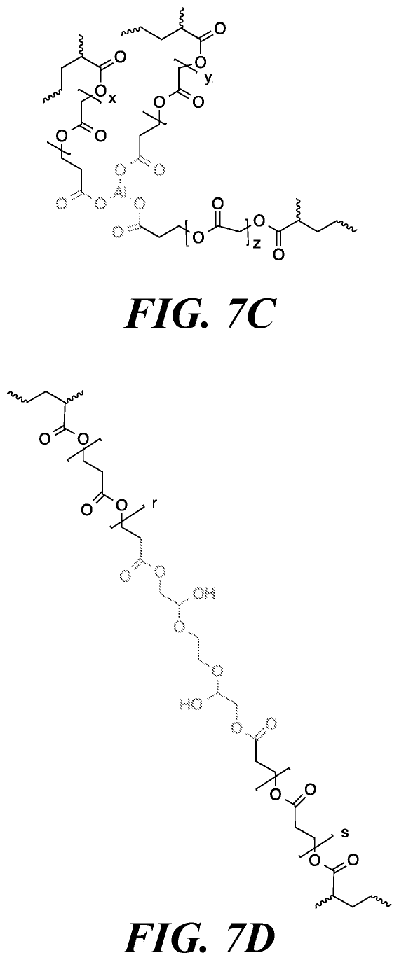

An example of a cross-linked polymer is depicted in FIG. 6. The type of cross-linking that occurs in the polymer depicted in FIG. 6 will depend on the types of cross-linker used to produce such polymer. For example, FIGS. 7A-7D depict various exemplary cross-linked polymers, including N,N′-methylenebis(acrylamide) (FIG. 7A), ethylene carbonate (FIG. 7B), aluminum acrylate (FIG. 7C), and ethylene glycol diglycidyl ether (FIG. 7D).

Molecular Weight

Molecular weight (including average molecular weight) and molecular weight distribution can be determined by any suitable methods or techniques known in the art.

In some embodiments, the polymer has a number average molecular weight of at least 1 million Daltons, at least 1.5 million Daltons, at least 2 million Daltons, at least 2.5 million Daltons, or at least 3 million Daltons; or between 1 million Daltons and 3 million Daltons, between 1 million Daltons and 2 million Daltons, or between 1 million Daltons and 1.5 million Daltons.

Particle Size and Particle Size Distribution

Particle size (including average particle size) and particle size distribution can be determined by any suitable methods or techniques known in the art.

In some embodiments, the polymer has an average particle size greater than 50 μm, greater than 55 μm, greater than 60 μm, greater than 65 μm, greater than 70 μm, greater than 75 μm, greater than 80 μm, greater than 85 μm, greater than 90 μm, greater than 95 μm, or greater than 100 μm; or between 50 μm and 500 μm, between 50 μm and 400 μm, between 50 μm and 300 μm, between 50 μm and 200 μm, between 50 μm and 150 μm, between 100 μm and 500 μm, between 200 μm and 500 μm, between 300 μm and 500 μm, or between 400 μm and 500 μm.

In other embodiments, the polymer has a particle size distribution between 50 μm and 900 μm, between 50 μm and 850 μm, between 50 μm and 700 μm, between 50 μm and 600 μm, between 50 μm and 500 μm, between 50 μm and 400 μm, between 50 μm and 300 μm, between 50 μm and 200 μm, between 50 μm and 150 μm, between 100 μm and 500 μm, between 200 μm and 500 μm, between 300 μm and 500 μm, or between 400 μm and 500 μm.

The particle size distribution may be described based on the distribution of more than 50%, 60%, 70%, 80% or 90% of particles. In some variations, the polymer has a particle size distribution of more than 50%, 60%, 70%, 80% or 90% of particles between 50 μm and 900 μm, between 50 μm and 850 μm, between 50 μm and 700 μm, between 50 μm and 600 μm, between 50 μm and 500 μm, between 50 μm and 400 μm, between 50 μm and 300 μm, between 50 μm and 200 μm, between 50 μm and 150 μm, between 100 μm and 500 μm, between 200 μm and 500 μm, between 300 μm and 500 μm, or between 400 μm and 500 μm.

In some aspects, provided are polymer compositions produced according to any of the methods described herein. The polymer compositions comprise any of the polymers described herein, and may further comprise residual monomers and extractables.

Residual Monomers

The residual monomer content may be of significant importance particularly for adsorbent polymers used in hygienic applications. For example, in some variations, the residual monomer content is the residual beta-propiolactone content, or the residual acrylic acid content, or a combination thereof. The residual acrylic acid may be derived from the beta-propiolactone.

The residual monomer content of the polymers described herein can be determined by any suitable methods or techniques known in the art. For example, high performance liquid chromatography (HPLC) may be used to quantify residual monomer.

In some variations, the polymer composition has a residual monomer content less than 1500 ppm, less than 1000 ppm, less than 900 ppm, less than 800 ppm, less than 700 ppm, less than 600 ppm, less than 500 ppm, less than 400 ppm, less than 300 ppm, less than 200 ppm, or less than 100 ppm.

Soluble Fraction or Extractables Content

Soluble fraction (sol) generally refers to the sum of all water-soluble species, including for example non-reacted starting materials and other residual monomers. Soluble fraction can be determined under any suitable methods or techniques known in the art. The sol content may be measured by extraction of a sample in water (e.g., distilled water), and the sol is often referred to in the art as “extractable”.

For example, in one variation, the soluble fraction can be measured by extraction of a sample in distilled water. A certain amount of the sample is poured into excess amount of water, and dispersed with magnetic stirring to reach equilibrium swelling. The swollen sample is filtered and dried. The sample weight loss results in the soluble fraction. See e.g., Zohuriaan-Mehr, M. J. and Kabiri, Kourosh, “Superabsorbent Polymer Materials: A Review”, Iranian Polymer Journal, 17 (6), 2008, 465.

In some embodiments that may be combined with the foregoing, the polymer composition has a soluble fraction of less than 20%, less than 15%, less than 10%, less than 5%, of less than 1% by weight of the polymer composition.

The polymer composition may also be described based on its extractables content. Extractables may include, for example, unreacted monomers and all other small molecules that are not the polymer. In some variations, the extractables content of the polymer composition may be expressed as follows:

Extractables content (weight %)=weight of extractable/(total weight of starting materials)

In some embodiments that may be combined with the foregoing, the polymer composition has an extractables content of less than 20%, less than 15%, less than 10%, less than 5%, of less than 1% by weight of the polymer composition.

Absorbency Under Load (AUL)

Absorbance generally refers to the amount of liquid that a material can hold. Absorbency under load generally refers to the absorbent capacity of a material, as measured under an applied load. Absorbency under load can be determined by any suitable methods or techniques known in the art. For example, in one variation, absorbance under load can be determined by scattering 0.2 g of a given absorbent material in an apparatus similar to a burette on a nonwoven fabric, and placing a load of 20 g/cm² in a cylinder and allowing artificial urine to be absorbed by the resin for 30 minutes. Such a test can determine the volume of artificial urine absorbed. Other methods known in the art to determine absorbency under load may be used. See e.g., Zohuriaan-Mehr, M. J. and Kabiri, Kourosh, “Superabsorbent Polymer Materials: A Review”, Iranian Polymer Journal, 17 (6), 2008, 463.

In some variations, the polymer or polymer composition has an absorbency under load of greater than 20 g/g, greater than 25 g/g, greater than 30 g/g, greater than 35 g/g, greater than 40 g/g, greater than 45 g/g, or greater than 50 g/g; or between 10 g/g and 50 g/g, between 10 g/g and 40 g/g, between 10 g/g and 25 g/g, between 20 g/g and 50 g/g, or between 25 g/g and 40 g/g.

In other variations, the polymer or polymer composition absorbs greater than 100 times, greater than 150 times, greater than 200 times, greater than 250 times, greater than 300 times, greater than 400 times, or greater then 500 times the dry weight of the polymer or polymer composition when contacted with a liquid. In yet other variations, the polymer or polymer composition absorbs between 100 times and 400 times, between 150 times and 400 times, or between 150 times and 300 times the dry weight of the polymer or polymer composition when contacted with a liquid.

Speed of Absorbance

Speed of absorbance refers to the rate at which a liquid is absorbed. Such liquid may be, for example, water. Speed of absorbance can be determined by any suitable methods or techniques known in the art. For example, in one variation, speed of absorbance can be determined by swelling kinetics methods. See, e.g., E. Southern, A. G. Thomas, Trans. Faraday Soc., 63, 1913 (1967).

In some variations, the polymer or polymer composition has a speed of absorbance greater than 10 g/g, greater than 15 g/g, or greater than 20 g/g; or between 10 g/g and 50 g/g, between 15 g/g and 50 g/g, between 15 g/g and 40 g/g, between 15 g/g and 30 g/g, or between 15 g/g and 20 g/g. In one variation of the foregoing, the speed of absorbance is measured at 0.3 psi at 5 min.

Swelling Capacity

Swelling capacity is a measure of absorbance. Swelling capacity may also be referred to in the art as “centrifuge retention capacity”. Swelling capacity can be determined by any suitable methods or techniques known in the art. See e.g., Zohuriaan-Mehr, M. J. and Kabiri, Kourosh, “Superabsorbent Polymer Materials: A Review”, Iranian Polymer Journal, 17 (6), 2008, 462-463. For example, in some variations, swelling capacity can be determined by the tea-bag method. A polymer sample may be placed into a tea-bag, and the bag is dipped in an excess amount of water or saline solution for one hour to reach the equilibrium swelling. The excess solution is removed by hanging the bag until no liquid is dropped off. The tea bag is weighed (W₁) and the swelling capacity is calculated according to the equation (1) below.

S_{c}=(W₁−W₀)/W₀ Equation (1)

Other methods known in the art may also be used to measure swelling capacity. In other variations, the centrifuge method may also be employed to measure swelling capacity. For example, 0.2 g (W₁) of the polymer sample is placed into a bag made of non-woven fabric. The bag is dipped in 100 mL of saline solution for half an hour at room temperature. Then, the bag is taken out, and then excess solution is removed with a centrifugal separator. Then, weight of bag (W₂) is measured. The same steps are carried out with an empty bag, and the weight of bag (W₀) is measured. The swelling capacity is then calculated by equation (2) below.

S_{c}=(W₂−W₀−W₁)/W₁ Equation (2)

In some embodiments that may be combined with the foregoing, the polymer or polymer composition has a swelling capacity of greater than 30 g/g, greater than 35 g/g, greater than 40 g/g, greater than 45 g/g, or greater than 50 g/g; or between 30 g/g and 50 g/g, between 30 g/g or 40 g/g, or between 30 g/g and 35 g/g.

It should generally be understood that any properties of the polymers or polymer compositions described herein may be combined as if each and every combination of the properties were individually listed. For example, in one variation, the polymer or polymer composition has: (i) an absorbency under load of between 12 g/g and 22 g/g; and (ii) a speed of absorbance of between 15 g/g and 20 g/g.

Bio-Content

In some variations of the foregoing, the polymer or polymer composition has a bio-content of greater than 0%, and less than 100%. In certain variations of the foregoing, the polymer or polymer composition has a bio-content of at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.9%, at least 99.99%, or 100%.

In some variations, bio-content (also referred to as “bio-based content”) can be determined based on the following:

% Bio-content or Bio-based content=[Bio(Organic)Carbon]/[Total(Organic)Carbon]*100%,

as determined by ASTM D6866 (Standard Test Methods for Determining the Bio-based Content of Solid, Liquid, and Gaseous Samples Using Radiocarbon Analysis).

The bio-content of the polymers or polymer compositions may depend based on the bio-content of the beta-propiolactone used. For example, in some variations of the methods described herein, the beta-propiolactone used to produce the polymers or polymer compositions described herein may have a bio-content of greater than 0%, and less than 100%. In certain variations of the methods described herein, the beta-propiolactone used to produce the polymers or polymer compositions described herein may have a bio-content of at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.9%, at least 99.99%, or 100%. In certain variations, beta-propiolactone derived from renewable sources is used. In other variations, at least a portion of the beta-propiolactone used is derived from renewable sources, and at least a portion of the beta-propiolactone is derived from non-renewable sources.

The bio-content of the beta-propiolactone may depend on, for example, the bio-content of the ethylene oxide and carbon monoxide used. In some variations, both ethylene oxide and carbon monoxide are derived from renewable sources.

With reference again to FIG. 4, when ethylene oxide 402 and carbon monoxide 404 are both obtained from renewable sources, beta-propiolactone 410 is bio-based. When such bio-based beta-propiolactone is polymerized according to the methods described herein, the resulting polymer is bio-based. For example, with references to FIGS. 1-3, when beta-propiolactone 102, 202, and 302 are produced from renewable sources, polymers 110, 210 and 310, respectively, are bio-based polymers.

Biodegradable

In some variations of the foregoing, the polymer or polymer composition has a biodegradability of at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.9%, at least 99.99%, or 100%.

In some variations of the foregoing, biodegradable is as defined and determined based on ASTM D5338-15 (Standard Test Method for Determining Aerobic Biodegradation of Plastic Materials Under Controlled Composting Conditions, Incorporating Thermophilic Temperatures).

Uses of the Absorbent Polymers

Diapers and Other Hygiene Products

In other aspects, provided herein are also absorbent articles comprising the polymers or polymer compositions described herein, or produced according to the methods described herein.

In some variations, the adsorbent article further includes at least one inorganic or organic additive. Suitable inorganic additives may include, for example, metals (such as aluminum or tin), as well as clays. The incorporation of such solids may enhance the absorbent properties of the polymer or polymer compositions. Examples of organic additives may include, for example, plasticizers such as polybutene, polypropene, polybutadiene, polyisobutene and/or polyisoprene.

In some embodiments, the absorbent article is a diaper, an adult incontinence product, or a feminine hygiene product. In some variations of the foregoing, the absorbent article is bio-based and/or biodegradable.

In certain aspects, provided is a biodegradable fabric, comprising any of the polymers or polymer compositions described herein, or produced according to the methods described herein. In some variations of the foregoing, the biodegradable fabric further comprises at least one inorganic or organic additive.

Agricultural Uses

The polymers or polymer compositions described herein, or produced according to the methods described herein, may also be suitable for agricultural use. In other aspects, provided is an agricultural product comprising the polymers or polymer compositions described herein, or produced according to the methods described herein. Such agricultural product may be a material used in the planting and/or growing of plants, or a seed or a crop.

For example, the polymers or polymer compositions described herein may be used as agricultural materials to hold water for crops. Thus, in some variations, provided is an agricultural material comprising the polymers or polymer compositions described herein. In certain variations, the agricultural material further includes at least one inorganic or organic additive.

In other variations, provided is a seed coated with the polymers or polymer compositions described herein. In other embodiments, provided is a seed mix comprising seeds, wherein at least a portion of the seeds is coated with the polymers or polymer compositions described herein. When the polymer or polymer compositions bio-degrade, water may be released.

In yet other aspects, provided is a method, comprising planting seeds, wherein at least a portion of the seeds is coated with the polymers or polymer compositions described herein. In some variations, the method further comprises growing plants from at least a portion of the planted seeds under conditions in which the polymers or polymer compositions bio-degrade to release water to the seeds and/or plants.

### EXAMPLES

The following Example is merely illustrative and is not meant to limit any aspects of the present disclosure in any way.

### Example 1

### Synthesis of Various Polymers, and Measurement of Water Absorbency

This Example demonstrates the synthesis of various polymers from beta-propiolactone (“bPL”). The water absorbency of these polymers were measured, and compared with the water absorbency of commercially available superabsorbent polymer produced from acrylic acid, purchased from Aldrich.

Polymer 1: bPL+10 Mol % NaAcr (No Crosslinker)

In a vial, 4.2 mmol of sodium acrylate and 42 mmol of bPL were added, and heated to 50° C. The temperature of the reaction was maintained at 50° C., until all the bPL was observed to be consumed.

Polymer 2: bPL+10 Mol % NaAcr+1 Mol % Ethylene Carbonate

In a vial, 4.2 mmol of sodium acrylate, 0.42 mmol of aluminum acrylate as a cross-linker, and 42 mmol of bPL were added, and heated to 50° C. The temperature of the reaction was maintained at 50° C., until all the bPL was observed to be consumed.

Polymer 3: bPL+10 Mol % NaAcr+1 Mol % Aluminum Acrylate

Polymer 3 was synthesized using a protocol similar to the one for polymer 2, except the cross-linker used was aluminum acrylate.

Polymer 4: bPL+10 Mol % NaAcr+1 Mol % Ethylene Glycol Diglycidyl Ether

Polymer 4 was synthesized using a protocol similar to the one for polymer 2, except the cross-linker used was ethylene glycol diglycidyl ether.

Polymer 5: bPL+10 Mol % NaAcr+N,N-Methylenebis(Acrylamide)

Polymer 5 was synthesized using a protocol similar to the one for polymer 2, except the cross-linker used was N,N-methylenebis(acrylamide).

Water Absorbency

The superabsorbent polymer (SAP) purchased from Aldrich, and the polymers synthesized in this Example were each tested for water absorbency using blue Dextran according to the protocols described in Fredric L. Buchholz, Journal of Chemical Education, Vol. 73, Number 6, p. 512. The water absorbency results are summarized in Table 1 below.

TABLE 1

| | Water |
|---|---|
| | Absorbency |
| Sample | (g/g) |
| SAP (Aldrich) | 134 |
| Polymer 1 (no cross-linker) | 14 |
| Polymer 2 (ethylene carbonate cross-linker) | 14 |
| Polymer 3 (aluminum acrylate cross-linker) | 1 |
| Polymer 4 (ethylene glycol diglycidyl ether cross-linker) | 6 |
| Polymer 5 (N,N-methylenebis(acrylamide) cross-linker) | 20 |

1. A method of producing a cross-linked polymer, comprising combining beta-propiolactone and a cross-linker in the presence of a metal cation to produce the cross-linked polymer,
wherein the cross-linked polymer comprises a partially neutralized polyacrylic acid backbone and a plurality of polypropiolactone side chains, and cross-linking moieties.

2. The method of claim 1, wherein the metal cation is provided as a metal salt.

3. The method of claim 2, wherein the metal is an alkali metal or an alkali-earth metal.

4. The method of claim 2, wherein the metal is sodium or potassium.

5. The method of claim 2, wherein the metal cation is provided as metal acrylate.

6. The method of claim 5, wherein the metal acrylate is sodium acrylate or potassium acrylate.

7. A method of producing a cross-linked polymer, comprising combining beta-propiolactone and a cross-linker to produce the cross-linked polymer,
wherein the cross-linked polymer comprises a partially neutralized polyacrylic acid backbone and a plurality of polypropiolactone side chains, and cross-linking moieties.

8. The method of claim 1, wherein the polypropiolactone side chains independently have a structure of formula —(CH₂CH₂(C═O)—O)ₙ⁻M⁺, wherein:
n is an integer from 1 to 10 inclusive; and
M⁺ is an alkali metal, a cross-linking moiety, or H⁺.

9. The method of claim 1, wherein the cross-linker comprises:
an acrylamide compound,
a metal acrylate compound,
an organic carbonate compound,
a diglycidyl compound, or
a vinyl-organic compound comprising two or more vinyl groups,
or any combination thereof.

10. The method of claim 1, wherein the cross-linker comprises ethyleneglycol dimethacrylate, diethyleneglycol diacrylate, allylmethacrylate, 1,1,1-trtimethylpropane triacrylate, triallylamine, or tetraallyoxyethane, or any combination thereof.

11. The method of claim 1, wherein the cross-linker comprises N,N′-methylenebis(acrylamide), aluminum acrylate, ethylene carbonate, and ethylene glycol diglycidyl ether, or any combination thereof.

12. The method of claim 1, wherein the cross-linker comprises a silane compound.

13. The method of claim 12, wherein the silane compound has a structure of formula Y₃SiR^{a}N⁺R¹R²R³X⁻, wherein:
Y is a hydrolyzable radical;
R^{a} is a divalent hydrocarbon radical;
each of R¹, R² and R³ is independently:
a saturated or unsaturated hydrocarbon radical, or
a saturated or unsaturated organic radical comprising carbon, hydrogen, and at least one heteroatom selected from the group consisting of oxygen, sulfur and nitrogen; and
X⁻ is an anion.

14. The method of claim 13, wherein R^{a} is a divalent hydrocarbon radical with 1 to 6 carbon atoms.

15. The method of claim 13, wherein each of R¹, R² and R³ is independently a saturated or unsaturated organic radical comprising (i) carbon, hydrogen and oxygen, (ii) carbon, hydrogen, and sulfur, or (iii) carbon, hydrogen and nitrogen.

16. The method of claim 13, wherein each of R¹, R² and R³ is independently a saturated or unsaturated organic radical consisting of (i) carbon, hydrogen and oxygen, (ii) carbon, hydrogen, and sulfur, or (iii) or carbon, hydrogen and nitrogen.

17. The method of claim 13, wherein X⁻ is chloride, bromide, fluoride, iodide, acetate or tosylate.

18. The method of claim 1, wherein the cross-linker comprises a polyol, a polyglycidyl ether, or a combination thereof.

19. The method of claim 1, wherein the cross-linker comprises a polysaccharide.

20. The method of claim 1, wherein the cross-linking moieties connect carboxylic end groups of at least a portion of the polypropiolactone side chains.

21. The method of claim 1, wherein the cross-linking moieties connect neutralized carboxylate groups of at least a portion of the polypropiolactone side chains.

22. The method of claim 1, wherein the cross-linking moieties connect at least a portion of the partially neutralized polyacrylic acid backbone.

23. The method of claim 1, further comprising combining the beta-propiolactone and the cross-linker with an ionic initiator, or a radical initiator, or a combination thereof.

24. The method of claim 23, wherein the ionic initiator comprises a salt of an alkali metal, a salt of an alkali-earth metal, or a combination thereof.

25. The method of claim 23, wherein the ionic initiator comprises a carboxylate salt of an alkali metal, a salt of an alkali-earth metal, or a combination thereof.

26. The method of claim 23, wherein the ionic initiator is a salt of an alkali metal.

27. The method of claim 23, wherein the ionic initiator has a structure of formula CH₂═CH₂CO₂⁻Z⁺, wherein Z⁺ is an alkali metal, an alkali earth metal, ammonium, a quaternary ammonium cation, or phosphonium.

28. The method of claim 27, wherein the quaternary ammonium cation is a lower alkyl quaternary ammonium cation.

29. The method of claim 23, wherein the ionic initiator is sodium acrylate, or potassium acrylate, or a combination thereof.

30. The method of claim 23, wherein the ionic initiator is a methacrylate.

31. The method of claim 23, wherein the ionic initiator is sodium methacrylate, or potassium methacrylate, or a combination thereof.

32. The method of claim 23, wherein the radical initiator comprises a peroxide, a persulfate, or an azo compound, or a combination thereof.

33. The method of claim 23, wherein the radical initiator is a redox initiator.

34. The method of claim 23, wherein the radical initiator comprises a hydroperoxide.

35. The method of claim 23, wherein the radical initiator comprises hydrogen peroxide.

36. The method of claim 1, further comprising combining the beta-propiolactone and the cross-linker with an additional monomeric compound.

37. The method of claim 36, wherein the additional monomeric compound is an organic compound comprising at least one vinyl group.

38. The method of claim 36, wherein the additional monomeric compound is methacrylic acid.

39. The method of claim 36, wherein the additional monomeric compound is an optionally substituted acrylic acid, or a carbohydrate, or any combination thereof.

40. The method of claim 1, further comprising carbonylating ethylene oxide to produce the beta-propiolactone.

41. The method of claim 1, further comprising combining ethylene oxide and carbon monoxide in the presence of a carbonylation catalyst and optionally a solvent to produce the beta-propiolactone.

42. A method of producing a cross-linked polymer, comprising: reacting a low molecular weight polypropiolactone with a radical polymerization initiator and a cross-linker,
wherein the low molecular weight polypropiolactone has a formula CH₂═CH₂—(C═O)—O—(CH₂CH₂(C═O)—O)ₙ⁻M⁺,
wherein n is an integer from 1 to 10 inclusive; and
M⁺ is an alkali metal, a cross-linking moiety, or H⁺.

43. A polymer produced according to the method of claim 1.

44. A polymer comprising a poly(sodium acrylate/acrylic acid) backbone and a plurality of polypropiolactone side chains connected to the backbone.

45. The polymer of claim 44, wherein the polymer is cross-linked.

46. A polymer comprising a partially neutralized polyacrylic acid backbone and a plurality of polypropiolactone side chains, and cross-linking moieties.

47. The polymer of claim 46, wherein the polypropiolactone side chains independently have a structure of formula —(CH₂CH₂(C═O)—O)ₙ⁻M⁺, wherein:
n is an integer from 1 to 10 inclusive; and
M⁺ is an alkali metal, a cross-linking moiety, or H⁺.

48. The polymer of claim 43, wherein the polymer has:
(i) a number average molecular weight over 1 million Dalton; or
(ii) an average particle size between 400 and 500 μm; or
(iii) a particle size distribution of more than 70% of particles between 300 μm and 600 μm; or
(iv) an extractables content less than 20%; or
(v) a residual monomer content less than 1500 ppm;
or any combination of (i) to (v).

49. The polymer of claim 43, wherein the polymer has:
(i) an absorbency under load between 10 g/g and 25 g/g; or
(ii) a speed of absorbance between 15 g/g and 20 g/g;
(iii) a swelling capacity between 30 g/g and 35 g/g; or
any combination of (i) to (iii).

50. The polymer of claim 43, wherein the polymer has:
an absorbency under load between 12 g/g and 22 g/g; and
a speed of absorbance between 15 g/g and 20 g/g.

51. The polymer of claim 43, wherein the polymer is bio-based as defined by ASTM D6866.

52. The polymer of claim 51, wherein the polymer has a bio-based content greater than 0% but less than 100%.

53. The polymer of claim 51, wherein the polymer has a bio-content of at least 20%.

54. The polymer of claim 43, wherein the polymer is biodegradable as defined by ASTM D5338-15.

55. An absorbent article, comprising a polymer of claim 43.

56. The absorbent article of claim 55, further comprising at least one inorganic or organic additive.

57. The absorbent article of claim 55, wherein the absorbent article is a diaper, an adult incontinence product, or a feminine hygiene product.

58. The absorbent article of claim 55, wherein the absorbent article is biodegradable.

59. A biodegradable fabric, comprising:
a polymer of claim 43; and
at least one inorganic or organic additive.

60. An agricultural product, comprising a polymer of claim 43.

61. The agricultural product of claim 60, wherein the agricultural product is a material to hold water for crops.

62. The agricultural product of claim 60, wherein the agricultural product is a seed or a crop.

63. A seed, wherein the seed is coated with a polymer of claim 43.

64. A seed mix, comprising a plurality of seeds, wherein at least a portion of the seeds is coated with a polymer of claim 43.

65. A method, comprising planting seeds of claim 63.

66. The method of claim 65, further comprising growing the seeds into plants under conditions suitable for the polymer to bio-degrade to release water to the seeds, the plants, or a combination thereof.
